(19) United States
(12) Patent Application Publication
(10) Pub. No.: US 2007/0173889 A1
(43) Pub. Date: Jul. 26, 2007

(54) Methods and apparatus for conversion of eukaryotic cells by application of electric and magnetic fields

(76) Inventors: Allen Rosenspire, Ann Arbor, MI (US); Howard R. Petty, Livonia, MI (US); Andrei L. Kindzelskii, Grosse Pointe Park, MI (US)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 10/585,677

(22) PCT Filed: Jun. 7, 2004
(86) PCT No.: PCT/US04/17931

§ 371 Date: Mar. 19, 2007

Related U.S. Application Data
(60) Provisional application No. 60/476,659, filed on Jun. 6, 2003.

(51) Int. Cl. A61N 1/00; C12N 15/00

(52) U.S. Cl. 607/2; 435/440

(57) ABSTRACT

The present invention comprises, without limitation, methods and apparatus to induce changes in eukaryotic cell shapes by application of low-level electric or magnetic fields and thereby to impact cell function or mobility. Thus, the present invention comprises methods and apparatus for the investigation and treatment of human and animal diseases, conditions, and defects.

This application claims priority from U.S. Provisional Patent Application No. 60/476,659, filed Jun. 6, 2003, which is hereby incorporated in full by reference. This invention was made in part during work partially supported by the U.S. government under NIH Grant No. AI27409 and CA74120O1. The government may have certain rights in the invention.

### FIELD OF THE INVENTION

The present invention relates generally to the field of the use of electrical and magnetic fields in biology.

### BACKGROUND OF THE INVENTION

Inflammatory responses are well-known causes or contributors to diseases and pathological conditions in humans and other animals. Mobility and migration of different cell types across and through cell junctions and into intercellular spaces are known phenomena that can affect the severity and timing of inflammatory diseases and conditions.

In similar fashion, mobility and migration of cells affect other diseases and conditions. As some examples, recruitment and migration of different cell types are important in tissue repair and regeneration. Similarly, migration of tumor cells affects the extent and severity of cancer in a host.

Many therapeutic treatments for such diseases and conditions involve the administration of drugs. However, in many instances, the ability to treat with drugs is limited, for example, due to the inability of the host to tolerate systemic side effects or due to the unavailability or ineffectiveness of drugs for a particular disease or condition.

Thus a significant need remains for methods and apparatus to affect the course of disease and pathological conditions, especially where the therapeutic use of drugs is limited, ineffective, or unavailable.

### SUMMARY OF THE INVENTION

In some embodiments, without limitation, the invention comprises the unexpected discovery that application of certain electric or magnetic fields produces changes and conversions of cell types such that the physiological function of the cells is modified in ways that may have important therapeutic significance.

Certaln eukaryotic cell types are known to demonstrate shapes that correspond with physiological function. As one example only, and without limitation, neutrophils are known to those of ordinary skill in the art to demonstrate shapes characterizable as “polarized” or “spherical.” Thus, to participate in an inflammatory reaction, immune cells, which are initially spherical in the circulation, must become polarized and motile as they extravasate from the circulatory system into the extracellular space. We have found that in vitro, polarized and motile immune cells are uniquely characterized by low frequency oscillatory behavior in their metabolism. In particular these cells exhibit characteristic oscillations in NAD(P)H metabolism. We have discovered unexpectedly that polarized neutrophils show fluctuations in internal NAD(P)H levels with periods of about 10 to 20 seconds at 37 degrees C. In contrast, this periodicity is not significant in spherical neutrophils. Analysis of NAD(P)H autofluorescence shows that its internal concentration fluctuates in a near sinusoidal pattem. Furthermore, in such cells, production of the inflammatory mediators nitric oxide (NO) and reactive oxygen species (ROS) is also periodic and closely coupled to the NAD(P)H oscillation.

As part of the present invention, we have discovered unexpectedly that weak, low frequency pulsed or sinusoidal electric fields can depolarize immune cells, thus substantially inhibiting the production of ROS and NO. In some embodiments, without limitation, this occurs after polarized cells are exposed to pulsed electric fields (for example, preferably about 10⁻⁴ volts/meter for certain cells) which coincide with NAD(P)H maxima, or to sinusoidal electric fields matched substantially in frequency and phase to the NAD(P)H oscillation. In some embodiments, without limitation, pulsed electric fields induced by weak magnetic fields are also effective in depolarizing immune cells. Thus, low intensity pulsed magnetic fields of chosen frequency range (in some embodiments, about 0.1 Hz) may terminate an inflammatory reaction in vivo, such as one occurring in an arthritic joint.

As part of the invention, we have discovered that when exposed to certain pulsed electrical or magnetic fields at a similar same frequency but not at the minima, polarized neutrophils show a collapse in their signature oscillation within a predictable time period. As a result, the polarized neutrophil depolarizes and assumes a spherical morphology. We have also found unexpectedly that exposure to sinusoidal electric fields that are substantially phase-matched to the oscillations can produce similar changes in morphology. This change in cell shape results in change in physiological function in the neutrophil, by way of example only, a decrease in mobility or migration across blood vessels during immunological responses.

The unexpected discovery of this novel method of manipulating cell shapes, and consequently, impacting cell function or mobility, has application across broad classes of eukaryotic cells, as examples only, among neutrophils, macrophages, lymphocytes, platelets, tumor cells, and retinal cells. By incorporation of this discovery, the present invention comprises methods and apparatus for the medical treatment of human diseases, conditions, and defects, for example and without limitation, in the ability to induce or effect therapeutic changes in blood or organ systems, tissue or wound repair or regeneration, immunological and inflammatory responses, cancers, developmental processes, and nerve regeneration. Thus, the invention comprises methods and apparatus applicable to broad fields of use, including without limitation, in the investigation and treatment of pathological conditions, diseases, or defects in animals and human beings.

### BRIEF DESCRIPTION OF THE DRAWINGS

The features and inventive aspects of the present invention will become more apparent upon reading the following detailed description, claims, and drawings, of which the following is a brief description.

FIG. 1 is a diagram showing the effect of nonresonant AC electric fields on neutrophil NAD(P)H oscillations and morphology.

FIG. 2 is a diagram showing the application of pulsed magnetic field at NAD(P)H minima that induces resonance in polarized neutrophils.

FIG. 3 is a chart showing electrical and magnetic characteristics of the coil and actuator utilized in FIG. 2.

FIG. 4 is a diagram illustrating the relationship between responsiveness of cells and time varying magnetic field.

FIG. 5 is a photograph illustrating the effects of magnetic fields on platelet aggregation.

FIG. 6 is a chart showing the measurement of NAD(P)H oscillations in individual neutrophils during various conditions.

FIG. 7 is a photograph of the appearance of neutrophils under various conditions.

FIG. 8 is a figure showing NAD(P)H oscillation in spherical and polarized neutrophils

FIG. 9 is a graph showing that pulsed DC electric fields resonate with naturally occurring NAD(P)H oscillations in human neutrophils.

FIG. 10 is a graph showing the application of a pulsed magnetic field at NAD(P)H minima or maxima.

FIG. 11 is a photograph showing the effect of pulsed magnetic fields on neutrophil morphology and adherence at a) 0 minutes exposure, b) 20 minutes exposure, or c) 22 minutes exposure.

### DETAILED DESCRIPTION

In some embodiments, the invention comprises methods and apparatus to mitigate or terminate the inflammatory response in disease conditions, such as joint inflammation found in arthritic patients. We have discovered unexpectedly that certain eukaryotic cells, such as neutrophils, which are both polarized and adherent, exhibit characteristic signature oscillations in their metabolism. In particular, we have discovered that internal NAD(P)H levels fluctuate in sinusoidal patterns with periods of approximately 10 or 20 seconds at 37 degrees C. Significantly, we have determined that in these cells, production of reactive oxygen species (“ROS”) and nitric oxide (“NO”) is directly proportional to the frequency and amplitude of the NAD(P)H oscillations. During inflammatory processes, polarized neutrophils are primary effector cells and through the generation of ROS and NO are responsible for much collateral tissue damage. In contrast, spherical neutrophils do not exhibit these low frequency NAD(P)H oscillations and do not produce significant quantities of ROS or NO.

In some embodiments, the invention comprises the unexpected discovery that, when a polarized eukaryotic cell, such as a neutrophil, is exposed to a weak, pulsed electric field of least 10⁻⁵ V/m, or in a preferred embodiment, around 10⁻⁴ V/m, of substantially the same frequency at its NAD(P)H oscillation, but which is delivered in such a way so as to not coincide with the NAD(P)H minima, the signature NAD(P)H oscillation will collapse within a detectable time period, for example, about one minute or so. Consequently the cell will depolarize and assume a spherical morphology, and its generation of ROS and NO will be greatly reduced. Thus, at least one embodiment of the invention comprises, without limitation, a method to deliver effective electrical pulses to populations of active ROS- and NO-generating neutrophils by magnetic induction, and in doing so, terminate or mitigate a neutrophil inflammatory response.

In some embodiments, the invention comprises methods and apparatus to treat inflammatory disease in humans through the local application of a time-varying magnetic field with the novel waveform which we have discovered. As one example only, an arthritic knee may be treated by attaching a magnetic coil to the outside of the knee and electronically activating the coil with an electrical current in order to produce the desired induced electrical signal within the knee joint space and induce the changes in cell shape that comprise part of the invention. Methods for calculation and application of such electrical signals are known to those of ordinary skill in the art, for example, see Buechler, et al., “Calculation of Electric Fields Induced in the Human Knee by a Coil Applicator,” Bioelectromagnetics 22:224 (2001), which is hereby incorporated by reference in full.

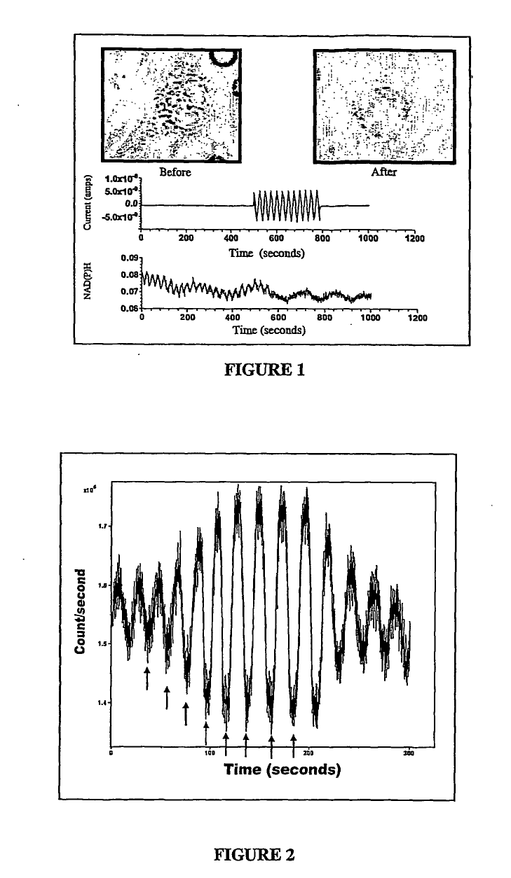

We have demonstrated that NAD(P)H levels in neutrophils and macrophages, as examples, are oscillatory. As one example only, we have also found that weak ultra low frequency AC of around at least 10-2 V/m or pulsed DC electric fields can resonate with, and increase the amplitude of NAD(P)H oscillations in these cells. We have discovered unexpectedly that externally applied AC fields can depolarize adherent neutrophils. FIG. 1 is a representative example of the effect of exposing polarized neutrophils to an AC field matched in frequency to the NAD(P)H frequency and shows an effect of non-resonant AC electric field on neutrophil NAD(P)H oscillations and morphology. A neutrophil which was polarized and adherent to a coverslip was selected, and NAD(P)H auto-fluorescence was monitored. After about 500 seconds, a low amplitude AC voltage (about 10⁻V/m) matched in frequency to the NAD(P)H oscillation was directly applied for about 100 seconds across the cell by platinum electrodes placed on the slide. The current and NAD(P)H signal were simultaneously recorded. After about 3 cycles the “20 second” NAD(P)H oscillation collapsed, leaving only the longer period oscillation intact. As indicated by the DISC images taken before and after application of the electric field, the collapse of the NAD(P)H oscillation was accompanied almost immediately by an extensive reorganization of the cytoskeleton, as the cell depolarized and assumed a spherical morphology. Although a sinusoidal electric field was used in this example, we have also found that polarized neutrophils will respond in a similar manner to pulsed electric fields as low as about 10⁻⁴ V/m, providing the pulses arrive at NAD(P)H maxima and minima. (Pulses which only arrive at the minima satisfy a resonance requirement, and augment NAD(P)H oscillations.)

For these cells, increased NAD(P)H amplitudes signal changes in behavior in the absence of cytokines or chemotactic factors. As one example only, we have studied the effect of pulsed DC electric fields on HT-1080 fibrosarcoma cells. As with neutrophils and macrophages, NAD(P)H levels oscillate. We found that weak (without limitation, about 10⁻⁵ V/m), but properly phased DC (pulsed) electric fields resonate with NAD(P)H oscillations in polarized and migratory, but not spherical, HT-1080 cells. In this instance, electric field resonance signaled an increase in HT-1080 pericellular proteolytic activity. Electric field resonance also triggered an immediate increase in the production of reactive oxygen metabolites. Thus, the ability of the invention's external electric fields to affect cell function and physiology by acting on NAD(P)H oscillations is not restricted to cells of hematopoietic lineage but may include many, if not all, polarized and migratory eukaryotic cells.

The existence of NAD(P)H oscillations, their linkage with the control of cell function, and their ability to resonate with external electric fields appear to be general phenomena in polarized migratory eukaryotic cells. Metabolic resonance and associated downstream effects do not appear to be restricted to neutrophils and macrophages, or other cells of hematopoietic lineage.

Application of extremely low frequency pulsed DC electric fields that are frequency- and phase-matched with endogenous metabolic oscillations leads to exaggerated neutrophil extension and metabolic resonance wherein oscillatory NAD(P)H amplitudes are increased. In particular, we have discovered that polarized, adherent and motile neutrophils, macrophages, and tumor cells, among others, exhibit a characteristic oscillation in their metabolism, such that the internal concentration of NAD(P)H fluctuates in a near sinusoidal pattern, with a period of approximately 20 seconds at 37 degrees C. In such cells, production of ROS and NO is also periodic, and is closely coupled to the NAD(P)H oscillation. We have previously shown that when these cells are exposed to either pulsed DC electric fields or AC electric fields as low as 10⁻⁵ to 10⁻⁴ V/m which are phase-matched to the natural NAD(P)H oscillation, that within a minute or so, the amplitude of the NAD(P)H oscillation, as well as the production of ROS and NO, increase several fold and remain elevated as long as the electric field application is continued. They then return rapidly to normal upon cessation of electric field exposure. This phenomena may be described as metabolic resonance.

As part of the invention we determined whether the metabolism of polarized neutrophils (NAD(P)H and production of ROS and NO) resonates with very weak pulsed ELF magnetic fields in a manner similar to the way that resonance with pulsed electric fields is established (e.g., FIGS. 2-4). Blood was drawn from healthy human donors, and neutrophils were immediately isolated by discontinuous density gradient centrifugation. The cells were suspended in Hank's Balanced Saline Solution (“HBSS”) and allowed to settle and adhere to a (cover) glass bottomed cell culture dish. The dish was then mounted on a heated stage held at 37 degrees C attached to an axiovert inverted fluorescence microscope (Zeiss). Using DIC microscopy, adherent polarized cells were selected. Utilizing epifluorescence microscopy, selected cells were individually illuminated, and NAD(P)H autofluorescence excited at 365 nm. NAD(P)H emission at wavelengths greater than 405 nm was recorded and analyzed in real time using a photomultiplier tube housed in a fluorescence microscope detection system interfaced with a computer. A coil placed directly over, and co-linear and centered with the culture dish, was transiently energized, and de-energized, in order to expose the cells to pulsed magnetic fields between zero and eight gauss. Coil activation and de-activation were linear with time and required 20 m sec. The effect of the magnetic pulses on NAD(P)H dynamics within an individual cell was then analyzed in real time. In addition to NAD(P)H, the effect of pulsed magnetic fields on the dynamics of pericellular ROS and NO production was also measured. This was accomplished with epifluorescence microscopy by incorporating ROS or NO sensitive fluorescent probes into the HBSS surrounding a cell, and then exciting the probes and analyzing their fluorescent emission at the appropriate wave lengths during exposure to pulsed magnetic fields.

We found that in adherent, polarized neutrophils, the metabolism resonated with external pulsed magnetic ,fields when the magnetic field pulses are delivered in such a way as to coincide with the minimum of the fundamental “20 second” NAD(P)H oscillation. NAD(P)H oscillatory amplitude, as well as pericellular ROS and NO production, increased so long as magnetic pulses continued to be delivered, but returned to normal levels when the pulses ceased. Furthermore, sensitivity to the magnetic pulses was a function of cellular distance from the center of the circular dish, suggesting that the cells were responding to induced electric field pulses rather than the magnetic fields per se. Calculations indicate that, in some embodiments, the minimum induced electric field intensity necessary for resonance is about 10⁻⁴ V/m.

FIG. 2 shows an application of a pulsed magnetic field at NAD(P)H minima induces resonance in polarized neutrophils. Polarized neutrophils responded to pulsed magnetic fields in a similar manner as to pulsed electric fields. Neutrophils were allowed to adhere and polarize on a 40 mm plastic tissue culture dish which was equipped with a 0.2 mm glass bottom. NAD(P)H auto-fluorescence from an individual cell was monitored as in the figures above. At the points indicated by the arrows, the cell was manually exposed to pulsed magnetic fields. Each pulse was generated by activating a coil placed 1.5 cm over, and co-linear with, the culture dish. The coil was activated with a linearly ramped current over 20 ms. In this experiment, without limitation, the maximum magnetic field at the center of the dish was about 2 gauss. The current was then held steady for about 200 ms, at which point the coil was deactivated in a linear fashion. This provided a square wave induced current of approximately 10⁻⁴ V/m in the vicinity of the cell, which was located towards the edge of the dish.

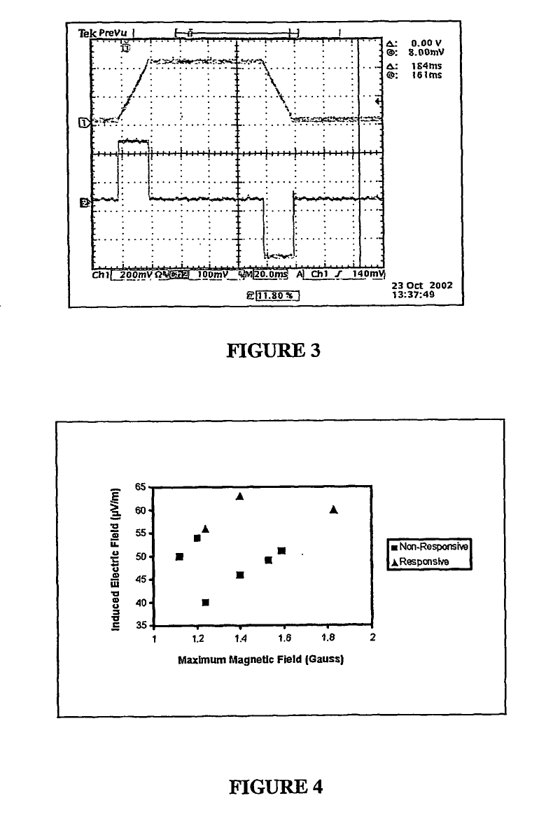

FIG. 3 is illustrative of the magnetic and electrical fields that the cells were exposed to in FIG. 2. FIG. 3 shows electrical and magnetic characteristics of the coil and actuator utilized in FIG. 2. (Traces 1-4 show a gauss field, 20 mS rise, 20 mS fall, linear slope. Trace 2 shows the induced field, via 7500:1 probe (small spikes in the chart are probe ringing, not signal.)) The first plot represents a recording of magnetic flux produced by the coil under test conditions. The linear rise and fall of magnetic flux ensures that induced electrical fields will be of a square wave nature. This is experimentally confirmed in the second plot, which is a recording of the induced electrical field, as directly measured with a probe.

FIG. 4 shows that the response of polarized neutrophils to a pulsed magnetic field is independent of the maximum value that the field obtains. In general we found that the responsiveness of cells to a time varying magnetic field is independent of the maximum value of the field, leading to the inference that the cells are responding to the induced electric field. In FIG. 4 neutrophils were allowed to adhere to the surface of a circular culture dish. Several different cells at varying distances from the center of the dish were chosen, and for each, the NAD(P)H oscillation was observed. Each cell was then exposed to a pulsed magnetic field of the waveform depicted in FIG. 3 above. In each instance the field reached its maximum value in 20 msec, but the maximum field strength itself was varied. The pulse was delivered at NAD(P)H minima. The cell either responded by resonance with the field as in FIG. 2, or it did not respond. The induced electric field in the vicinity of each cell was proportional to the rate of change of the magnetic field multiplied by the distance of the cell from the center of the culture dish. Therefore, in each instance, the induced electric field was calculated and recorded, along with the maximum value that the magnetic field obtained, and cellular response (or lack of response). The data is plotted in FIG. 4, where a response or not for each cell is noted along with induced electric field strength and maximum magnetic field strength. It is clear that whether or not a cell responds to a pulsed magnetic field is independent of the final magnetic field strength. Response is solely dependent upon the induced electric field surpassing a critical threshold of about 56 μV/m.

Thus, the invention comprises the discovery that the metabolism of neutrophils dramatically responds to extremely low intensity and ultra low frequency (as one example only, about 0.05 Hz) pulsed electric fields, whether the fields are directly applied or induced by time-varying magnetic fields. These fields may also be utilized to signal cellular metabolism much as cytokines or drugs. Because magnetic fields easily penetrate tissue, our discoveries confirm that ultra low frequency pulsed magnetic fields may be useful to control cellular metabolism in vivo, with concomitant therapeutic value.

In many cases, animal and human cells which are initially non-polarized must transform into a polarized phenotype in order to accomplish particular biological functions. For instance, in order to fully participate in an inflammatory reaction, at some point immune cells which are initially non-polarized and essentially spherical in the circulation (e.g., neutrophils, macrophages, or lymphocytes) must become adherent, assume a polarized morphology, and become motile. Of significance is the fact that such polarized cells are sensitive to externally applied weak electric fields, in the sense that if the cell is exposed a periodic (sinusoidal or pulsed) electric field of the proper strength, frequency and phase, the immune cells will revert from a polarized morphology back into a spherical (and non-inflammatory) morphology.

The minimal electric field intensities necessary to initiate the transformation are generally very weak, and, in some embodiments, without limitation, are of the order of about 10⁻⁴ volts/meter for pulsed electric fields, and about 10⁻² volts/meter for sinusoidal fields. For each polarized cell, the appropriate frequency and phase characteristics which an electric field must possess in order to effect the transformation from a polarized to a spherical morphology is determined by analysis of the cell's metabolism. In particular, NAD(P)H is an important and abundant intermediate cellular metabolite, and its autofluorescence can be utilized by methods known in the art to monitor the metabolism of an individual cell with the use of an epifluorescence microscope. In polarized, adhered, and motile cells, NAD(P)H levels were found to vary in a sinusoidal fashion, with a period inversely dependent on temperature. For neutrophils, we find average periods of about 21 and 19.5 seconds for temperatures of about 37 and 39 degrees C, respectively. In the presence of endotoxin or cytokines such as IL2, the cells become activated, and the periods are decreased to about 11 and 10 seconds. The results for other polarized cell types are similar.

In some embodiments, in order to transform a polarized, adhered, and motile cell into a spherical cell, the cell is exposed to either pulsed or sinusoidal electric fields. When sinusoidal electric fields are used in some embodiments, the frequency of the field must be adjusted to within about 10% of the frequency of the NAD(P)H oscillation, and minimum field strength of about 10⁻² volts/meter achieved. The ability of a sinusoidal field to promote conversion into a spherical phenotype is generally independent of phase, but generally it is necessary for the electric field to be applied for a length of time equal to about at least three to four periods (τ) of the initial cellular NAD(P)H oscillation.

We discovered that pulsed electric fields are also effective in transforming polarized cells. In some embodiments, when pulsed fields are utilized, it is necessary that irregardless of the exact shape of the pulse, that a field intensity of at least approximately 10⁻⁴ volts/meter across the cell be achieved for no less than about 20 milliseconds during each pulse. Succeeding pulses should be delivered at a frequency within about 5-10% of either 1×, 2×, or 4× that of the NAD(P)H frequency. We found that there is rather broad latitude in the permissible phase relationship between the NAD(P)H oscillation and the pulsed electric field. However, if a frequency equal to about that of the NAD(P)H oscillation is used, pulses cannot be coincident with the NAD(P)H minima, nor can they be delivered near (within about ±π/10 radians) of a minima. Whether a pulse frequency of about 1×, 2×, or 4× the NADP)H frequency is used, successive pulses may, or may not, be of the same electrical polarity.

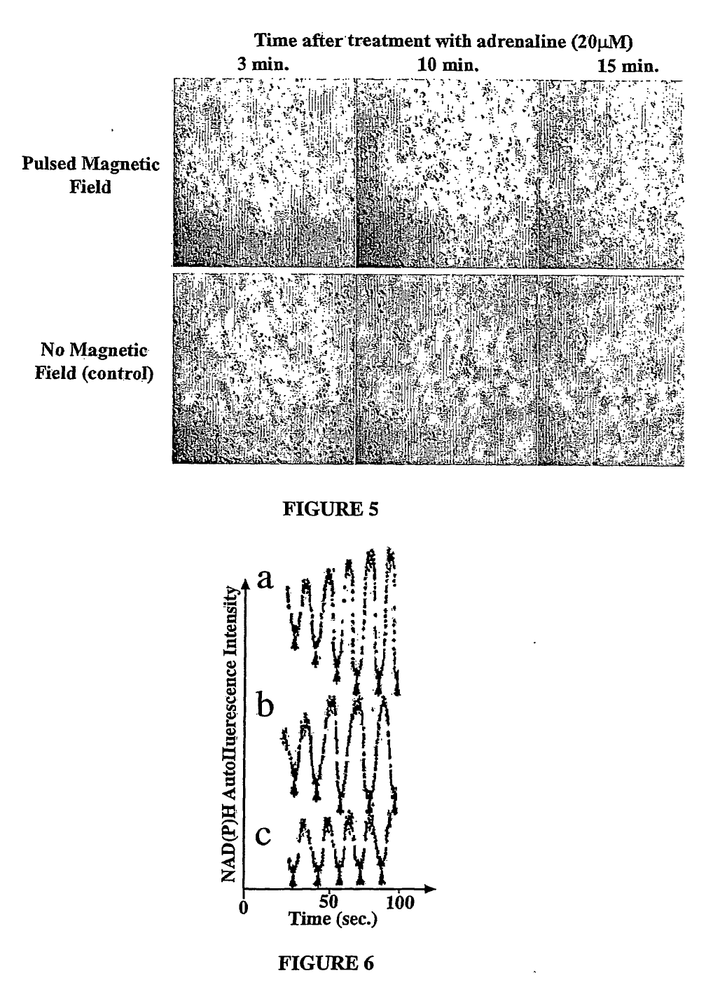

In some embodiments, the invention comprises methods and apparatus to affect platelet aggregation and influence blood clotting in therapeutic applications. Human platelets treated with adrenaline form cell aggregates. This is a model of platelet aggregation in vivo, such as that which occurs during thrombus formation during heart attacks, strokes, and many other cardiovascular disorders. An electric field was applied to platelets, as described above for neutrophils. In FIG. 5, aggregation is illustrated in the absence of an applied magnetic field, as disclosed herein. However, in the presence of a magnetic field, platelet aggregation is not observed. Therefore, in some embodiments, application of magnetic fields of the invention at the appropriate timing characteristics influences the ability of platelets to aggregate and therefore magnetic fields may be applied locally to influence blood clotting in various tissues.

As part of the invention, electrical fields, whether sinusoidal or pulsed, may be applied to cells directly by placing chemically inert (e.g., platinum) electrodes in the vicinity of the cells, and then applying the voltage output of an electrical waveform generator directly to the electrodes. Cells may also be effectively exposed to electric fields which are locally induced in the vicinity of the cell by the application of a time varying magnetic field.

In some embodiments, either of these methods can be applied to depolarize cells in vitro, as well as within tissues in vivo. However, electric fields may be severely attenuated within animal tissues, while the same tissues are relatively transparent to magnetic fields. Thus for the application of pulsed or sinusoidal electric fields to depolarize cells which are embedded within tissues, magnetic induction is a preferred method, without limitation. Furthermore, because the magnitude of any induced electric field would necessarily be proportional to the rate of change of the applied magnetic field, and considering that the period necessary for an induced periodic electric field (sinusoidal or pulsed) to effectively depolarize cells is on the order of about 10 to 20 seconds, induction of pulsed fields is preferable to induction of sinusoidal fields.

In some embodiments of the invention, the method (of magnetic induction of ultra low frequency pulsed electric fields) can be used to terminate or mitigate an inflammatory reaction, as one example only and without limitation, one that takes place in an arthritic human knee. This is so because in order to participate in an inflammatory response (such as one characterizing an arthritic knee), inflammatory cells (e.g., neutrophils, macrophages or lymphocytes) which participate in the response, will have had at some time to transition to a polarized morphology in order to extravasate from the circulatory system into the knee joint space, and functionally participate in the inflammatory reaction. However, if the volume surrounding the knee joint space is permeated by a magnetically induced periodic electric field of the type disclosed herein as effective in depolarizing inflammatory cells, then inflammatory cells will be prohibited from participating in the inflammatory reaction, and the inflammatory reaction and consequent physiological damage will necessarily be reduced. In some embodiments, in order to induce pulsed electric fields in a tissue, a coil applicator would be placed over the region of interest. For instance a coil applicator designed specifically for the human knee is described in Buechler, supra. In some embodiments, the coil may be energized by a saw tooth wave form, but the coil may also be energized with an electric current that increases in a linear fashion to its maximum value over about 20 millisecond interval. In some embodiments, without limitation, the coil current then remains approximately constant at its naximum value (as does the magnetic field produced by the coil) for a period of time greater than about 20 milliseconds, but less than about 0.5 seconds, before decreasing to about zero in a linear fashion over the following about 20 millisecond interval. Under this scenario, independent of the magnitude of the peak current and magnetic field, the coil applicator will induce in the knee a pair of (about 20 millisecond duration) symmetrical square wave electrical pulses of opposite polarity separated by about 20 to about 500 milliseconds

In either embodiment, and without limitation, whether the coil is activated by a saw tooth wave form, or one which induces symmetrical square waves, a critical parameter is that the electric field pulses induced by the coil in the knee cartilage, whatever their shape, meets or exceeds an intensity of about 10⁻⁴ volts/meter for at least about 20 milliseconds. By utilizing algorithms known to those of ordinary skin in the art, the peak magnetic field (and hence peak current through the coil applicator) may be calculated with some precision so as to ensure that the minimum intensity of the induced electric pulses within the tissue is at least about 10⁻⁴ volts/meter.

As part of some embodiments of the invention, in order to depolarize a cell characterized by an NAD(P)H oscillation of period r seconds with a pulsed electric field, it is necessary that after successive periods of either about τ, τ/2, or τ/4 seconds from the beginning of the first induced pulse, that the coil applicator be reenergized, and de-energized for several cycles. In general the electrical pulses should continue for a length of time equal to at least about 5 ×τ. If the electrical pulses are of about the same frequency as the NAD(P)H oscillation, then it is required that the initial pulse be phased so as to not occur within about τ/10 seconds of an NAD(P)H minima. The phase relationship between the NAD()H oscillation and the induced electrical pulses is not critical when the electrical pulses are characterized by periods of about τ/2, or τ/4 seconds.

In vitro τ and phase may be determined for individual cells directly by observation of NAD(P)H autofluorescence. However this is usually not possible iin vivo, as would be the case for inflammatory cells within a knee, for instance. We have found, however, that at about 37 degrees C, the average metabolic period for a non-activated, but otherwise polarized, adherent, and motile cell is about 21 seconds, with a standard deviation of approximately 10%. Furthermore the average NAD(P)H period itself is a function of temperature, which in inflamed tissue is not fixed, but can be expected to vary by several degrees centigrade over time and location. The situation is further complicated by the fact that inflammatory cells may likely be characterized as a population of cells with random NAD(P)H phase relationships. Therefore, in some embodiments, without limitation, within a tissue environment such as the knee, no single frequency and phase can be chosen for an applied pulsed electric field, which will suffice to depolarize all inflammatory cells at once. However, a series of at least about 11 pulse trains with periods τ=about 17, 18, 19, . . . 24 seconds, may overlap the metabolic frequencies of all non-activated inflammatory cells within a normal or inflamed tissue, with a greater than about 95% degree of confidence.

When frequencies approximating the metabolic frequencies of inflammatory cells are chosen for the pulse trains, there are likely to be some cells for which an electrical pulse arrives at or near the NAD(P)H minima. Under these conditions, the cell will not depolarize. Therefore, in some embodiments, to ensure depolarization of the broadest spectrum of cells, for each discrete frequency, a minimum of about 10 pulse trains, each differing in phase by at least about π/5 radians, should be utilized.

The invention also comprises the use of electrical pulse trains characterized by frequencies which are about 2× or 4× the expected cellular metabolic frequencies. Considering that normally most polarized, but non-activated, cells within an inflamed tissue should be characterized by metabolic oscillations between about 17 and 24 seconds, in some embodiments, without limitation, it is necessary to employ either a series of about 11 pulse trains with periods τ=about 8.5, 9, 9.5 . . . 12 seconds, or τ=about 4.25, 4.5, 4.75 . . . 3 seconds. With these higher frequency pulse trains, a specific phase relationship between the electrical pulses and the metabolic oscillations is no longer necessary. For this reason it is preferable to utilize electrical pulses at frequencies which are about 2× or 4× those of the expected metabolic frequencies.

As mentioned above, it can be expected that non-activated polarized cells within an inflamed tissue may have metabolic oscillations with periods averaging between about 21 and 19.5 seconds. The disclosure herein indicates how pulsed electric (or alternatively pulsed magnetic) fields can be designed to depolarize cells with about these metabolic frequencies. However, when a tissue is inflamed, the internal cytokine profile is altered, and as a result it is likely that many polarized cells within the tissue become “activated” so as to be characterized by lower metabolic periods of about 10 to 11 seconds. In order to ensure that these cells are also depolarized, it is necessary that they be exposed to pulsed electric fields matched to about their frequencies.

The invention comprises methods and apparatus to depolarize these “activated” cells in a manner analogous to the one described above for cells with periods between about 21 and 19.5 seconds. In particular, in some embodiments, pulsed magnetic fields induced by a coil which is linearly energized over about a 20 millisecond period, and then de-energized over about 20 milliseconds, 20 to 500 milliseconds later is effective. However we have found that on average, “activated” immune cells have metabolic periods which are decreased by a factor of about 1.9 from cells which are not activated, and this must be taken into account. This means that, in some embodiments, if pulse trains with periods of about 17-24 seconds are initially used to depolarize non-activated inflammatory cells within a tissue, then pulse trains with periods of about 8.9-12.6 seconds should also be utilized to depolarize the activated cells. Pulse trains with periods of about 2×, or 4× the NAD(P)H fundamental frequencies, are also effective in depolarizing cells. Because activated cells have a frequency approximately 2× that of the non-activated cells, we have found that pulse trains with periods of about 4.2 to 6 seconds are effective at depolarizing both activated as well.as non-activated cells. Furthermore issues of phase relationship between the pulsed electric fields and NAD(P)H oscillations are unimportant at these frequencies. Use of about 10 sequential electrical pulse trains, induced by pulsed magnetic fields, encompassing periods between about 4 to 8 seconds, is a preferred method to prevent polarized immune cells from contributing to an inflammatory reaction within a tissue.

Once polarized cells are depolarized by electrical fields, it is possible that after a period of about 10 to 15 minutes that they may begin to re-polarize. Therefore, in some embodiments, after an initial sequence of magnetic or electrical pulses, it is necessary that the pulse sequence be repeated with a time delay of under about 10 minutes. In some embodiments, pulsed fields may have to be continuously applied until underlying damage within inflamed tissue is repaired.

Possible mechanisms for the conversion of cell shapes were also evaluated, including without limitation, the roles of membrane potentials and calcium detection of ultra-low frequency pulse electric fields. By way of example, only, we have investigated the possible role of ion channels in the detection of extremely low frequency electric fields by neutrophils. Human peripheral blood neutrophils were obtained and exposed to pulsed ELF electric fields. Weak DC electric fields were briefly applied about every 20 sec. such that the timing of field application (or phase) corresponded to certain intracellular chemical oscillators. In our experiments Kv1.3 and Ca2+ channels were unlabeled, labeled with fluorescent anti-Kv1.3 or DM-bodipy (−)-dihydropyridine (FL-DHP), respectively, or blocked with various channel blockers. High-speed imaging was performed by methods known to those of ordinary skill.

We discovered that neutrophils with Kv1.3 and Ca2+ channel clusters, which were morphologically polarized, responded to weak electric fields. However, spherical cells, which display randomly distributed channels, did not sense the presence of electric fields. Cells responded by increasing their length and by increasing the amplitude of intracellular NAD(P)H oscillations (e.g., FIG. 6), reflecting metabolic resonance. FIG. 6 shows the measurement of NAD(P)H oscillations in individual neutrophils during various conditions. Panel a of FIG. 6 shows untreated cells exposed to phase-matched electric field (arrows) which promotes enhanced amplitudes of the NAD(P)PH oscillations. Using a low dose of verapamil, which does not affect potassium channels, increased amplitudes are observed. However, the reagent mibefradil blocked these changes at pharmacologically relevant doses.

Specifically, fluorescence resonance energy transfer experiments of neutrophils labeled with both anti-Kv1.3 and FL-DHP reagents showed that these labels were in close proximity (less than or equal to about 7 nm), suggesting that the channels may be in physical contact. Dose-response analyses of the effects of the channel blockers tetraethylammonium (TEA), 4-aminopyridine (4-AP) and verapamil on metabolic resonance were consistent with a role of K+ channels in field detection. However, the time required for inhibition of metabolic resonance (about 25 min.) suggested that K+ channels may not be the primary field detector. Since Kv1.3 is a principal K+ leak channel in hematopoietic cells, the effect of K+ channel blockers are likely explained by their ability to reduce the neutrophil's membrane potential.

A panel of Ca+2 channel blockers was also evaluated. Only mibefradil was found to block metabolic resonance at a pharmacologically-relevant concentration. This indicates that T-type Ca+2 channels, which are functional only in a narrow, low voltage range, participate in electric field detection. A role of the calcium signaling apparatus is also suggested because: 1) 8-Br-cADPR, an inhibitor of CD38, blocks metabolic resonance at physiological doses and 2) electric fields alter intracellular Ca2+ signaling in indo-1-labeled neutrophils as indicated by both quantitative microfluorometry and high-speed imaging of Ca2+ signals.

These results suggest that when the transmemnbrane potential reaches a critical value, an applied weak pulsed electric field promotes T-type Ca+2 channel signaling, which is observed as a discrete calcium wave with high-speed microscopy. Thus, in some embodiments of the invention, the timing of the applied electric field will coincide with peak of the transmembrane potential, thus accounting for the phase dependence. Furthermore, channel clusters were observed. The close physical proximity of the channels suggests that additional amplification of the signal may be mediated by cooperative interactions among the channels within a cluster.

We also evaluated the “optical” transmembrane potential (“OTP”) of neutrophils, characterizing temporal oscillations and identifying membrane domains with elevated potentials and potassium leak currents. As demonstrated by us, migrating neutrophils exhibit numerous endogenous oscillations including without limitation, calcium signaling, metabolism, membrane potential, cytoskeletal assembly, receptor associations, shape change, NO and superoxide production. Because weak electric fields matching the period of these endogenous oscillations affect neutrophil properties and functions, we characterized the endogenous transmembrane potential of these cells.

Human peripheral blood neutrophils were obtained, then exposed to pulsed ELF electric fields as described Weak DC electric fields were applied approximately every 20 sec. such that the timing of field application (or phase) corresponded to certain intracellular chemical oscillators. OTPs were detected using the dye di-8-ANEPPS according to methods know to those of ordinary skill in the art. K+ leak sites were observed with the fluorescent K+ indicator PBFI in a 1% gelatin solution in which the cations were replaced with tetramethylammonium. High-speed imaging was performed as described in Kindzelslii, et al., “Apparent role of traveling metabolic waves in periodic oxidant release by living cells”, Proc. Natl. Acad. Sci., USA 2002 99, 9207-9212, which is hereby incorporated by reference.

Quantitative microfluorometry revealed that the neutrophil's OTP oscillated with a period of about 20 sec., although additional frequency components of lower intensities were noted. The OTPs were rising sawtooth waves with a periodic depolarization. As expected, the oscillatory amplitude disappeared in the presence of about 400 mM K+. Addition of oubain, an inhibitor of the Na+/K+-ATPase, reduced the slope but not the period of the oscillations. The application of exogenous pulsed electric fields at the OTP oscillation peak enhanced the depolarization. This is not likely due to a direct field effect on the OTP, but instead an indirect effect on cation currents as suggested by the ability of channel blockers to inhibit the enhanced depolarization. The effect could not be explained by photobleaching since the fluorescence intensity returned to a normal oscillatory pattern when the field was discontinued. Fluorescence microscopy experiments indicated that the OTP was much higher at the leading edge of the cell than at the uropod. When the OTP was evaluated using high speed microscopy, periodic depolarizations, which appeared as dark bands traveling around the cell perimeter at about 160 microns/sec., were found. Since the K+ leak current is primarily responsible for setting the membrane potential, we evaluated this current using an extracellular K+ indicator in a gelatin matrix to finish the diffusion of the indicator and K+. Migrating neutrophils demonstrated large amounts of K+ near the lamellipodium, thus accounting for the local amplitude of the OTP.

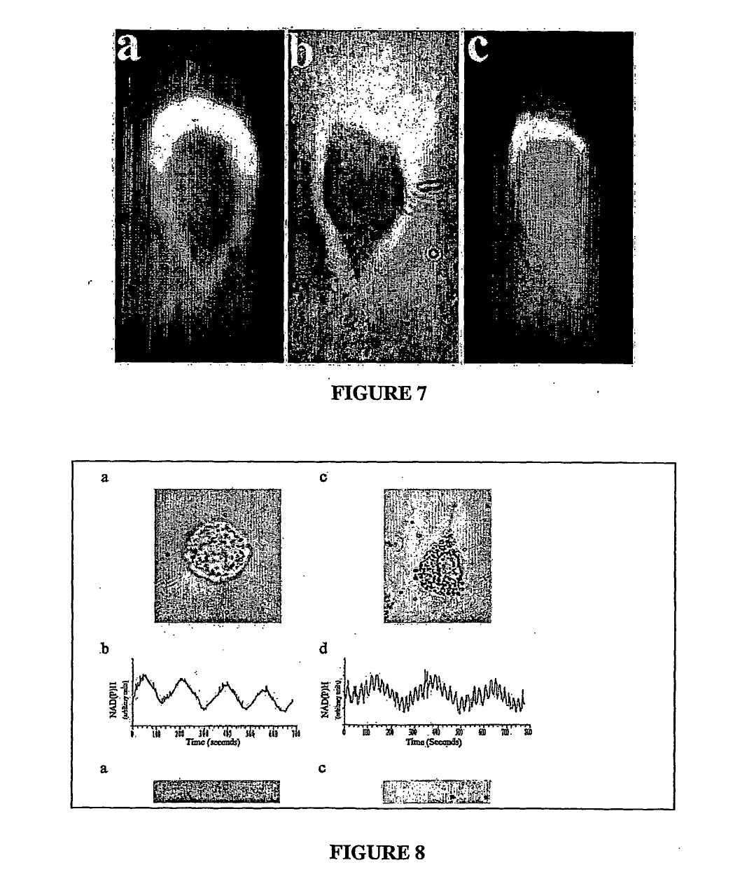

Since Kv1.3 is a principal K+ leak channel in hematopoietic cells, the distribution of Kv1.3 was studied using immunoflourescence microscopy (see FIG. 7). FIG. 7 illustrates the appearance of neutrophils during (a) membrane potential visualization; (b0 the leakage of K+ into the extracellular environment; and (c) labeling with a fluorescent anti Kv1.3 antibody. The direction of migration is toward the top of FIG. 7. We demonstrated that Kv1.3 was extensively clustered at the lamellipodium, thus accounting for enhanced K+ leak.

Thus, some embodiments of the invention comprise methods to analyze the temporal and spatial features of the OTP of small living cells. The membrane potential has numerous vectorial features on migratory cells, which were previously unrecognized. Furthermore, the OTP is affected by weak ELF fields whose phases are matched to the oscillatory potential, but the effect is likely an indirect one affecting cation currents.

Osteoarthritis (OA) is a disorder involving hyaline cartilage and subchondral bone, where all tissues in and around the involved joint are hypertrophic. The etiology of OA is understood to essentially involve repetitive mechanical stress leading to joint failure. It is the most common of the articulation disorders, and is often seen in knee joints by age 70, although pathological changes by age 40 are not uncommon. Because collagen, like bone has piezoelectric properties, and pulsed electromagnetic fields have been shown to stimulate chrondocytes to proliferate or increase synthesis of proteoglycans, the successful application of exogenous pulsed electromagnetic fields in treating nonunion fractures has prompted investigations into the use of pulsed electromagnetic fields to treat osteoarthritis. Randomized double blind placebo controlled clinical trials have recently established therapeutic benefit of pulsed electromagnetic fields in osteoaruitis of the knee and cervical spine. As in bone, there is no clear understanding of the frequency response, but frequencies in the vicinity of 15 Hz seem to be most effective at stimulating chrondocytes.

Rheumatoid arthritis (RA) is a systemic autoimmune disorder of unknown etiology whose major distinctive feature is chronic erosive synovitis of peripheral joints. In the United States the prevalence of RA is 0.3% in persons under age 35, but increases to over 10% over age 65. As opposed to OA, investigations into the use of electromagnetic fields to treat RA are far fewer in number. There have been some recent clinical studies of limited extent, which have shown that static magnetic fields may be of some benefit in the treatment of RA, as well as one report that a sinusoidal magnetic field at a frequency of 100 Hz was also useful. However the populations are small, and there is no theoretical reasoning presented as to why RA should respond to either static or 100 Hz magnetic fields. In contrast, our work indicates that ultra-low frequency, pulsed magnetic fields, as one example only, in the frequency domain of 0.05 to 0.1 Hz, should prove effective in the treatment of RA.

Although the etiology of RA is unknown, substantial progress has been made in understanding the molecular and cellular aspects of RA pathogenesis. In particular, it is now clear that neutrophil and macrophage accumulation in RA synovial fluids are characteristic pathological events, and that within an RA joint, both of these cell types are highly activated. Activated neutrophils release proteinases, prostaglandins, leukotrienes, NO, and ROS, all of which directly contribute to joint inflammation and injury. Activated macrophages produce an extensive list of their own inflammatory mediators, including IL-1α and IL-1β, TNF-α, platelet derived growth factor (PDGF), heparin binding (fibroblast) growth factors (FGF), and transforming growth factors (TGF). These cytolones stimulate resident joint synovial fibroblasts and chondrocytes to produce and release still other products, including collagenase and transin/stromelysin, which directly contribute to joint destruction. We have found that pulsed electromagnetic fields may be utilized to specifically interfere with the pathogenic mechanisms. Specifically, we have discovered that pulsed magnetic fields may be utilized to specifically deactivate neutrophils and macrophages within an RA joint, as well as to prevent any further recruitment of inflammatory cells into the joint from the blood stream.

In order to participate in an inflammatory response in an RA joint, immune cells (neutrophils, macrophages, lymphocytes), which in the circulation are essentially spherical, have to transform into a polarized morphology, so as to extravasate from the circulatory system into the joint space. Furthermore, once in the joint, these cells retain the polarized morphology which is associated with an activated phenotype. As noted herein, we have found that polarized (as opposed to spherical) immune cells exhibit a characteristic oscillation in their metabolism, such that the internal concentration of NAD(P)H fluctuates in a near sinusoidal pattern, with a period of approximately 20 seconds at 37° C. In such cells, production of ROS and NO is also periodic, and is closely coupled to the NAD(P)H oscillation, so that the production of these reactants is significantly higher in polarized as opposed to spherical cells. Significantly, we have also found that when polarized cells are exposed to pulsed DC electric fields of about 10⁻⁴ V/m which are of the same frequency, (but 180° out of phase with the natural NAD(P)H oscillation), that within a minute or so, the amplitude of the NAD(P)H oscillation, as well as the production of ROMs and NO increase several fold.

On the other hand, if polarized immune cells are exposed to either pulsed or sinusoidal electric fields which are in phase with the NAD(P)H oscillation, within a minute of so, the NAD(P)H oscillation will collapse. The collapse of the NAD(P)H oscillation will immediately be accompanied by a substantial decrease in pericellular ROM and NO production, and within several minutes cells will revert from the polarized activated phenotype back into the spherical morphology characteristic of inactive circulating cells. Thus it appears that exogenously applied weak ultra-low frequency pulsed or sinusoidal electric fields can be utilized to tap into immune cell signal transduction pathways in order to reliably control their behavior in real time. Significantly we have found inz vitro that we can also control immune cell behavior with the application of ultra-low frequency pulsed magnetic fields. The pulsed magnetic fields indirectly interact with the immune cells through the induction of local electric fields.

Thus magnetic induction of ultra low frequency pulsed electric fields should be useful in terminating an inflammatory reaction, as might take place in an RA joint. This will occur because in order to participate in such a reaction, inflammatory cells will have to transition to a polarized morphology in order to extravasate from the circulatory system into the joint space, and functionally participate in the inflammatory reaction. However, if the volume surrounding the joint space is permeated by a magnetically induced periodic electric field of the type described above as effective in depolarizing inflammatory cells, then inflammatory cells within the joint which are actively participating in the reaction will be inhibited. Furthermore, recruitment of additional inflammatory cells will be prohibited, so that as a whole, the inflammatory reaction and consequent physiological damage will necessarily be reduced.

In accordance with the invention, we have demonstrated NAD(P)H oscillations in spherical and polarized neutrophils. See FIG. 8, which shows that NAD(P)H oscillates in spherical and polarized neutrophils. Neutrophils were suspended in Hank' balanced salt solution, placed on a slide, covered with a coverslip, and then mounted on a heated (37° C.) microscope stage. FIG. 8a shows a spherical neutrophil that was selected and imaged by differential interference contrast microscopy. FIG. 8b shows the time dependence of NAD(P)H within the spherical cell that was determined by monitoring NAD(P)H autofluorescence by epifluorescence microscopy. NAD(P)H oscillates with a period of about 180 sec. FIG. 8c shows a polarized and adherent neutrophil that was selected and imaged by differential contrast microscopy. FIG. 8d shows the time dependence of NAD(P)H within the polarized cell that was determined by monitoring NAD(P)H autofluorescence by epifluorescence microscopy.

FIG. 8a is a representative example (n=100) of a differential interference contrast (DIC) image of a nonpolarized spherical neutrophil. After the DIC image was recorded, the cell was illuminated at 365 um, and the resulting fluorescence was collected above 405 nm with epifluorescence microscopy. The fluorescence intensity of the entire cell is plotted as a function of time in FIG. 8b (τ=180 s). It has been previously shown that under these conditions fluorescence is due almost exclusively to. NAD(P)H. Because cytosolic NAD(P)H concentration is strongly coupled to cellular metabolism, epifluorescence microscopy of intracellular NAD(P)H opens a real-time window on metabolic activity at the single-cell level. In this case NAD(P)H oscillations with a period of ˜3 min are suggestive of an underlying metabolic oscillation with a similar period.

Metabolic oscillations in polarized and adherent cells appear to be more complex than those of spherical cells, in that a second higher frequency oscillation mode appears. FIG. 8c is a DIC image of a representative example (n=100) of a polarized neutrophil that has adhered to a coverslip, and for which NAD(P)H fluorescence was assessed as in FIG. 8b. In this instance we find (FIG. 8d) that superimposed upon the “long” period oscillation (about 230 s for this cell) is an oscillation with a period of 21.6 s.

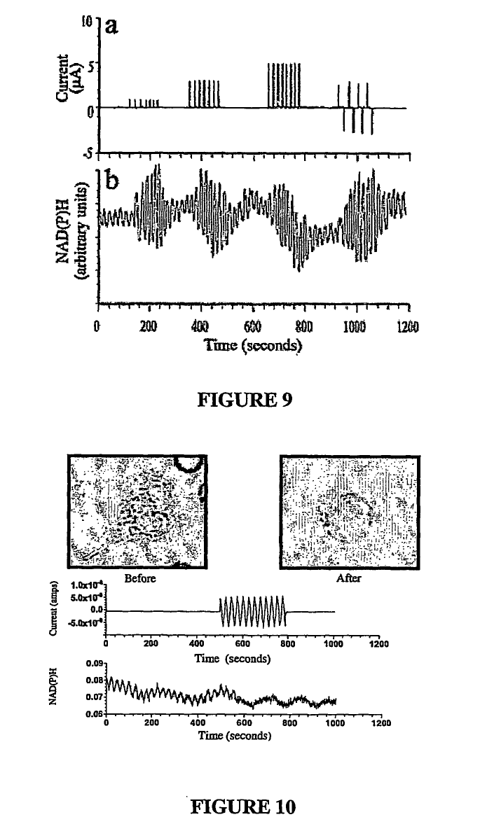

Metabolic oscillations can be controlled by externally applied pulsed DC electric fields with either uniform or alternating polarity. FIG. 9 shows that pulsed DC electric fields resonate with naturally occurring NAD(P)H oscillations in human neutrophils. Neutrophils were placed in the cell chamber described below and allowed to settle and adhere to the coverslip for 20-30 min. The chamber was mounted on a heated 37° C. microscope stage, a neutrophil was selected, and the autofluorescence arising from NAD(P)H was measured. Square-wave DC pulses were then applied across the chamber. Each pulse was timed to coincide with a successive NAD(P)H minimum. FIG. 9a shows currents resulting from successive application of pulse chains of 1, 5, and 10 V of uniform polarity, followed by a fourth pulse chain of 5 V with alternating polarity, plotted as a function of time. FIG. 9b shows the corresponding trace for the cellular NAD(P)H autofluorescence plotted along the same time scale.

FIG. 9 shows a representative example (n=100), where an individual neutrophil was allowed to adhere to and polarize on a glass coverslip, and NAD(P)H fluorescence was monitored as described above. After the first few NAD(P)H cycles, an electric field generated by a square wave voltage source was synchronously applied across the cell at NAD(P)H minima. In the absence of an applied voltage, NAD(P)H concentration oscillates uniformly with a period of about 20 s. However, after application of only the second voltage pulse, NAD(P)H began to resonate with the electric field, in that the amplitude clearly began to increase. The NAD(P)H amplitude reached a maximum value after the sixth pulse. When the voltage pulses ceased, the NAD(P)H amplitude returned to baseline values. After a brief period, resonance was then reestablished as a series of additional synchronous voltage pulses was administered. However, in this case, while the applied voltage was increased by a factor of 5 over the initial pulses, the maximum NAD(P)H amplitude remained unchanged. When the field was terminated, the NAD(P)H amplitude again rapidly decayed to baseline values. A third series of synchronous pulses was then administered, but at 10 times the initial field intensity. Once again resonance was quickly established, with the NAD(P)H oscillations having the same maximum amplitude as before, indicating that the resonance is an all-or-none phenomenon. The NAD(P)H amplitude again decayed to baseline values when field pulses were discontinued. Finally, a fourth series of synchronized pulses was applied, but this time field polarity was reversed on alternate pulses. We found that resonance was established just as rapidly, and in fact it appears to be indistinguishable from the resonance induced by previous uniform pulse trains. Thus it appears that polarity is unimportant in the ability of an external pulsed electric field to resonate with neutrophil metabolism, as long as each pulse is still timed to coincide with an NAD(P)H mininum.

We have also discovered that polarized neutrophils respond to pulsed magnetic fields in a similar manner as to pulsed electric fields. In FIG. 10a, neutrophils were allowed to adhere and polarize onto a glass surface. NAD(P)H auto-fluorescence from an individual cell was monitored as in the figures above. At the points indicated by the arrows in FIG. 10 (local minima in NAD(P)H), the cell was manually exposed to pulsed magnetic fields. Each pulse was generated by activating a coil placed 1.5 cm over, and co-linear with, the surface. The coil was activated with a linearly ramped current over 20 ms. In. this experiment, the maximum magnetic field that the cells were exposed to was about 2 gauss. The current was then held steady for about 200 ms, at which point the coil was deactivated in a linear fashion. This provided a square wave induced current of approximately 10⁻⁴ V/m in the vicinity of the cell. In general the cell responds in an analogous manner as was seen in the response to the pulsed electric fields in FIG. 9, in that the NAD(P)H oscillation increases in amplitude so long as the pulses continue to be ap plied, but returns to normal when they cease, a phenomena which we have referred to as metabolic resonance. In FIG. 10b, neutrophils were allowed to adhere to a glass surface, NAD(P)H concentration monitored, and magnetic pulses delivered all as in FIG. 10a. However in this figure, the magnetic pulses were delivered (points indicated by the arrows) at times which coincided with NAD(P)H maxima in the target neutrophil. It can be seen that unlike FIG. 10a, that under these conditions, application of the magnetic pulses quickly resulted in the collapse of the. NAD(P)H oscillation. Note that the time scale in FIG. 10a is about double that of FIG. 10b).]

FIG. 3 is illustrative of the magnetic and electrical fields that the cells were exposed to in FIG. 10. The first plot represents a recording of magnetic flux produced by the coil under test conditions. The linear rise and fall of magnetic flux ensures that induced electrical fields will be of a square wave nature. This is experimentally confirmed in the second plot, which is a recording of the induced electrical field, as directly measured with a probe.

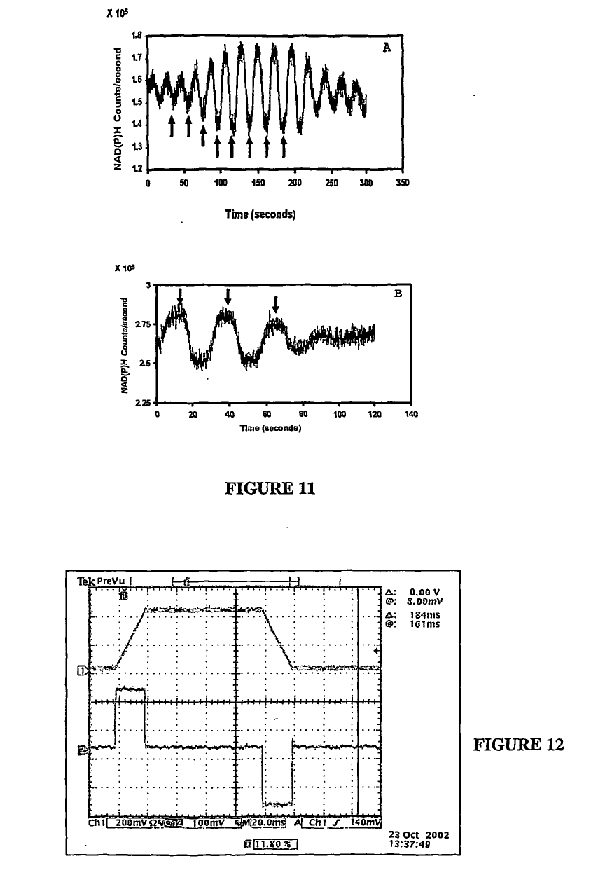

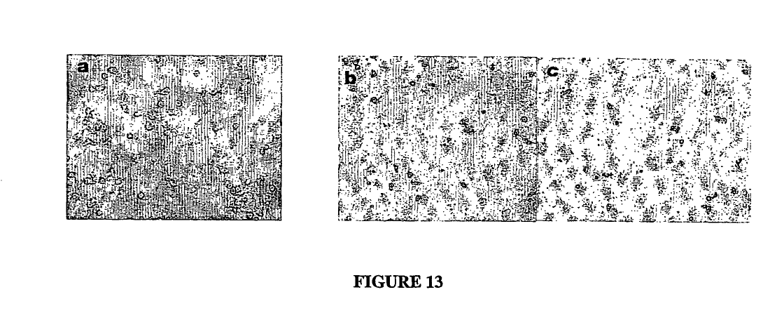

In accordance with some embodiments of the invention, without llmitation, a simple paradigm for the application of externally applied pulsed magnetic fields may suffice to depolarize an arbitrary population of adherent neutrophils in the absence of detailed knowledge of precise NAD(P)H frequencies and phase relationships between individual cells. FIGS. 1 and 10 demonstrate that given a neutrophil's NAD(P)H frequency and phase, sufficient information is available to guide the application of either a sinusoidal AC electric field, or a pulsed magnetic field, so as to depolarize the cell. However, if pulsed magnetic fields are to be utilized to depolarize neutrophils and other polarized inflammatory cells in vivo, as one example only and without limitation, within an arthritic knee, then it is unlikely that one would have knowledge of the NAD(P)H frequencies and phases of each inflammatory cell. Nevertheless, FIG. 11. demonstrates that, in some embodiments of the invention, even in the absence of such detailed knowledge, the current level of understanding of the effect of electromagnetic fields on neutrophil metabolic oscillations is sufficient to design and apply a protocol for the application of magnetic pulses which will be effective in depolarizing an arbitrary population of randomly phased cells.

As only one example, and without limitation, in FIG. 11, as in FIGS. 1 and 8, normal human neutrophils were allowed to settle and adhere onto a coverslip. The coverslip was mounted on an inverted microscope equipped with a temperature controlled stage held at 37° C., and without benefit of first observing cellular NAD(P)H, the cells exposed to a series of magnetic pulses generated utilizing a computer to control the coil and actuator described in FIG. 3. Initially a pulse train consisting of 6(0.5 second) pulses, gated at an arbitrary period between 9 and 11 seconds was generated. This was followed by an identical pulse train, but delayed in phase by two seconds. The process was repeated an additional 2 times, so that in total 24 pulses were delivered. At this point, the computer randomly selected a second gating interval between the limits of 9 and 11 seconds, and another 24 pulses were generated with the second gating frequency. Pulse trains at randomly selected gating frequencies between the limits of 9 and 11 seconds continued to be generated, and the cells exposed to the pulsed magnetic fields for 22 minutes.

FIG. 11a is an image of the cells prior to application of magnetic fields. Note that most of the neutrophils present as adherent, flattened, and polarized. (For the most part the spherical cells visible in the field are contaminating red cells). FIG. 11b is an image of the same cell preparation, but after 20 minutes of magnetic pulse exposure. The figure demonstrates that relatively few of the neutrophils are flattened and/or polarized. Also, essentially all of the cells in the field have lost adherence, even those that still maintain a partially polarized morphology. This point is shown in FIG. 11c. FIG. 11c represents the same field of view as FIG. 11b, but was obtained 2 minutes later. Because of cell movement into and out of the visual field, the distribution of cells in FIG. 11b is markedly different from that of FIG. 11c. Control experiments demonstrate that over a 22 minute interval under identical conditions, neutrophils which are not exposed to magnetic pulses maintain their initial polarized morphology, and do not detach from the coverslip (not shown).

Although certain preferred embodiments of the present invention have been described the invention is not limited to the illustrations described and shown herein, which are deemed to be merely illustrative of the best modes of carrying out the invention. A person of ordinary skill in the art will realize that certain modifications and variations will come within the teachings of this invention and that such variations and modifications are within its spirit and the scope as defined by the claims.

What is claimed is:

1. A method of converting the shape of polarized eukaryotic cells, comprising the steps of:
determining a frequency of internal NAD(P)H oscillation within the polarized eukaryotic cells, and
applying an electric field of sinusoidal frequency to the cells.

2. The method of claim 1, wherein the electric field has a frequency within about ten per cent of the frequency of internal NAD(P)H oscillation within the cells.

3. The method of claim 1, wherein the field strength of the electric field is at least 10⁻² volts/meter.

4. The method of claim 1, wherein the frequency of the electric field is substantially phase-matched to the frequency of internal NAD(P)H oscillation within the cells.

5. The method of claim 1, wherein the electric field is applied to the cells for a time period equal to at least three periods of the internal NAD(P)H oscillation.

6. The method of claim 1, wherein the electric field is applied by means of magnetic induction.

7. A method of converting the shape of polarized eukaryotic cells, comprising the steps of:
determining a frequency of internal NAD(P)H oscillation within the polarized eukaryotic cells, and
applying an electric field of sinusoidal frequency to the cells, wherein:
the electric field has a frequency within ten percent of the frequency of internal NAD(P)H oscillation within the cells;
the frequency of the electric field is substantially phase-matched to the frequency of internal NAD(P)H oscillation within the cells; and
the field strength of the electric field is at least 10⁻² volts/meter.

8. The method of claim 7, where the electric field is applied to the cells for a time period equal to at least three periods of the internal NAD(P)H oscillation of the cells.

9. The method of claim 7, wherein the polarized eukaryotic cells are selected from the group consisting of neutrophils, macrophages, lymphocytes, platelets, tumor cells, and retinal cells.

10. A method for converting the shape of polarized eukaryotic cells, comprising the steps of:
determining a frequency of internal NAD(P)H oscillation within the polarized eukaryotic cells, and
applying a pulsed electric field to the cells.

11. The method of claim 10, wherein the electric field has a frequency within ten percent of the frequency of the internal NAD(P)H oscillation frequency within the cells.

12. The method of claim 10, wherein the field strength of the electric field is at least 10⁻⁵ volts/meter.

13. The method of claim 10, wherein the field strength of the electric field is at least 10⁻⁴ volts/meter.

14. The method of claim 10. wherein the electric field is applied at other than the minima of the NAD(P)H oscillation frequency.

15. The method of claim 10, wherein the electric field is applied by means of magnetic induction.

16. A method for converting the shape of polarized eukaryotic cells, comprising the steps of:
determining a frequency of internal NAD(P)H oscillation with the polarized eukaryotic cells, and
applying a pulsed electric field to the cells, where:
the electric field has a frequency within ten percent of the frequency of the internal NAD(P)H oscillation within the cells, and
the field strength of the electric field is at least 10⁻⁵ volts/meter.

17. The method of claim 16, where the field is applied at other than the minima of the internal NAD(P)H oscillation.

18. The method of claim 16, where the field is applied at other than about ±π/10 radians of the internal NAD(P)H oscillation.

19. The method of claim 16, wherein the electric field is applied by means of magnetic induction.

20. The method of claim 16, wherein the polarized eukaryotic cells are selected from the group consisting of neutrophils, macrophages, lymphocytes, platelets, tumor cells, and retinal cells.

21. A method of mitigating an inflammatory condition in a mammal, comprising the step of:
applying an electric field of sinusoidal frequency to a tissue comprising an inflammatory condition of the mammal, wherein the field strength of the electric field within tissue comprises at least 10⁻² volts/meter.

22. The method of claim 21, wherein the electric field is applied by means of magnetic induction.

23. The method of claim 22, wherein the means of magnetic induction comprises a coil applicator.

24. The method of claim 21, wherein the mammal is a human.

25. A method of mitigating an inflammatory condition in a mammal, comprising the step of:
applying a pulsed electric field to a tissue comprising an inflammatory condition of the mammal, wherein the field strength of the electric field within tissue comprises at least 10⁻⁵ volts/meter.

26. The method of claim 25, wherein the electric field is applied by means of magnetic induction.

27. The method of claim 26, wherein the means of magnetic induction comprises a coil applicator.

28. The method of claim 26, wherein the magnetic induction comprises a time-varying magnetic field.

29. The method of claim 28, wherein the magnetic field comprise a square wave form.

30. The method of claim 28, where the magnetic field comprises a sawtooth wave form.

31. The method of claim 25, wherein the mammal is a human.

32. A method of mitigating an inflammatory condition in a mammal, comprising the steps of:
applying a pulsed electric field to a tissue comprising an inflammatory condition of the mammal, wherein:
the field strength of the electric field within tissue comprises at least 10⁻⁵ volts/meter, the electric field is applied by means of magnetic induction, and
the magnetic induction comprises a time-varying magnetic field.

33. The method of claim 32, wherein the means of magnetic induction comprises a coil applicator that is transiently energized by activation and deactivation of the coil applicator with electric current in a linear manner.

34. The method claim 32, wherein the pulsed electric field is comprised of at least one pulse train of at least two pulses.

35. The method of claim 32, wherein the mammal is a human.

36. A method of treating a pathological condition in a mammal, comprising the step of:
applying a pulsed electric field to a region of the mammal's body comprising the pathological condition of the mammal, wherein:
the field strength of the electric field within tissue comprises at least 10⁻⁵ volts/meter, the electric field is applied by means of magnetic induction, and
the magnetic induction comprises a time-varying magnetic field.

37. The method of claim 36, wherein the mammal is a human.

38. A method of converting the shape of polarized eukaryotic cells, comprising the steps of:
applying an electric field to the polarized eukaryotic cells by means of magnetic induction, wherein the magnetic induction is comprised of at least two pulse trains each comprised of at least two magnetic pulses.

39. The method of claim 38, wherein at least one pulse train is delayed in phase from at least one previous pulse train.

40. The method of claim 39, wherein at least one pulse train is gated for a period of between 9 and 11 seconds.

41. The method of claim 38, wherein a first pulse train is comprised of at least six pulses gated for a period of between 9 and 11 seconds, and a second pulse train of at least six pulses is delayed in phase from a previous pulse train.

42. A method of treating a pathological condition in a mammal, comprising the step of:
applying an electric field to the polarized eukaryotic cells by means of magnetic induction, wherein the magnetic induction is comprised of at least two pulse trains each comprised of at least two magnetic pulses.

43. The method of claim 42, wherein a first pulse train is comprised of at least six pulses gated for a period of between 9 and 11 seconds, and a second pulse train of at least six pulses is delayed in phase from a previous pulse train.
